(19) United States
(12) Patent Application Publication
(10) Pub. No.: US 2024/0082569 A1
(43) Pub. Date: Mar. 14, 2024

(54) DEVICES, SYSTEMS, AND METHODS FOR TREATING DISEASE USING ELECTRICAL STIMULATION

(71) Applicant: Phagenesis Limited, Manchester (GB)

(72) Inventor: Conor Mulrooney, Manchester (GB)

(21) Appl. No.: 18/463,061

(22) Filed: Sep. 7, 2023

Related U.S. Application Data
(60) Provisional application No. 63/375,603, filed on Sep. 14, 2022.

(51) Int. Cl. A61N 1/05

(52) U.S. Cl. CPC A61N 1/0526

(57) ABSTRACT

Electrical stimulation devices and associated systems and methods are disclosed herein. In some embodiments, the electrical stimulation device comprises an elongated member configured to be orally or nasally inserted into a patient's upper gastrointestinal tract. The device may further include a conductive element carried by a distal portion of the elongated member and configured to deliver stimulation energy to nearby tissue to treat disorders of consciousness.

### CROSS-REFERENCE TO RELATED APPLICATIONS

This application claims the benefit of priority to U.S. Provisional Application No. 63/375,603, filed Sep. 14, 2022, which is incorporated herein by reference in its entirety.

### TECHNICAL FIELD

The present disclosure is directed generally to devices, systems, and methods for treating disorders of consciousness using electrical stimulation.

### BACKGROUND

Disorders of consciousness are characterized by alterations in wakefulness and/or awareness, and common causes of disorders of consciousness include cardiac arrest, traumatic brain injury, intracerebral hemorrhage, and ischemic stroke. Several kinds of brain injury can cause disorders of consciousness, including diffuse bihemispheric lesions, bilateral lesions within the rostral paramedian brainstem, bilateral diencephalon lesions with unilateral brainstem involvement, and/or metabolic or toxic encephalopathies that produce widespread dysfunction of the corticothalamic system and its connections with the basal ganglia and limbic system. Regardless of etiology, the common pathophysiological mechanism underlying disorders of consciousness is broad withdrawal of excitatory synaptic activity across the cerebral cortex. Disorders of consciousness comprise a broad spectrum of alterations in wakefulness and/or awareness that can be characterized as coma in which there is a complete absence of wakefulness and awareness, unresponsive wakefulness syndrome in which there is wakefulness without awareness, minimally conscious state in which there is wakefulness and minimal, reproducible but inconsistent awareness, and confusional state in which there is wakefulness and awareness but persistent dysfunction across multiple cognitive domains, behavioral dysregulation, disorientation, etc.

Disorders of consciousness cause great human suffering and material costs for society. In addition to the immediate health and quality of life concerns introduced by disorders of consciousness, acute complications and chronic medical comorbidities are highly prevalent with patients of disorders of consciousness. As but one example, patients with disorders of consciousness often suffer from dysphagia and have difficulty swallowing or are unable to swallow safely. Complications that have been associated with dysphagia include pneumonia, malnutrition, dehydration, poorer long-term outcome, increased length of hospital stay, increased rehabilitation time and the need for long-term care assistance, increased mortality, and increased health care costs. These complications impact the physical and social wellbeing of patients, quality of life of both patients and caregivers, and the utilization of health care resources.

Existing treatments for disorders of consciousness suffer from limited effectiveness and/or are highly invasive. Accordingly, there remains a need for improved devices and methods for treating disorders of consciousness.

### SUMMARY

The present technology relates to electrical stimulation devices and associated systems and methods. In particular embodiments, the present technology comprises electrical stimulation devices configured to perform electrical stimulation of a patient's upper gastrointestinal tract to treat disorders of consciousness. The subject technology is illustrated, for example, according to various aspects described below, including with reference to FIGS. 1-4. Various examples of aspects of the subject technology are described as numbered clauses (1, 2, 3, etc.) for convenience. These are provided as examples and do not limit the subject technology.

1. A method for increasing a consciousness level of a patient, the method comprising:
via a nasal or oral cavity, positioning a conductive element of a stimulation device in a lumen of an upper gastrointestinal tract of a patient;
stimulating a sensory nerve proximate the lumen; and
in response to stimulating the sensory nerve, improving a consciousness level of the patient.

2. The method of Clause 1, wherein stimulating the sensory nerve excites central nervous system regions associated with consciousness.

3. The method of any one of the preceding Clauses, wherein the sensory nerve includes the vagus nerve.

4. The method of any one of the preceding Clauses, wherein the sensory nerve includes the glossopharyngeal nerve.

5. The method of any one of the preceding Clauses, wherein the sensory nerve comprises a sensorimotor nerve.

6. The method of any one of the preceding Clauses, wherein stimulating the sensory nerve drives organizational changes and/or excitability level changes in the brain.

7. The method of any one of the preceding Clauses, wherein improving a consciousness level of the patient comprises improving at least one of eye opening, verbal response, motor response, or autonomic response of the patient.

8. The method of any one of the preceding Clauses, wherein positioning the conductive element of the stimulation device in the lumen of the upper gastrointestinal tract of the patient comprises positioning the conductive element of the stimulation device in a lumen of a pharynx of the patient.

9. The method of any one of the preceding Clauses, wherein positioning the conductive element of the stimulation device in the lumen of the pharynx of the patient comprises positioning the conductive element within the oropharynx and/or the laryngopharynx.

10. The method of any one of the preceding Clauses, wherein positioning the conductive element of the stimulation device in the lumen of the pharynx of the patient comprises positioning the conductive element in apposition with a posterior wall of the pharynx.

11. The method of any one of the preceding Clauses, wherein positioning the conductive element of the stimulation device in the lumen of the pharynx of the patient comprises positioning the conductive element in apposition with at least one of an anterior wall of the pharynx, a lateral wall of the pharynx, or a medial wall of the pharynx.

12. The method of any one of the preceding Clauses, wherein the stimulation device includes a feeding tube having an extracorporeally positioned proximal end and a distal end positioned within a gastrointestinal tract of the patient, and wherein the distal end of the feeding tube remains positioned in the patient's gastrointestinal tract while stimulating the sensory nerve.

13. The method of any one of the preceding Clauses, wherein stimulation energy delivered to the sensory nerve has a frequency between about 1 Hz and about 200 Hz, an amplitude between about 1 mA and about 50 mA, and a pulse width between about 200 μS and about 1 mS with a total energy delivered to the sensory nerve during a treatment session being less than 8 J.

14. The method of any one of the preceding Clauses, wherein stimulation energy delivered to the sensory nerve has a frequency between about 1 Hz and about 200 Hz, an amplitude between about 1 mA and about 50 mA, and a pulse width between about 200 μS and about 1 mS with a total energy per pulse delivered to the sensory nerve being less than 3 J.

15. The method of any one of the preceding Clauses, wherein stimulation energy delivered to the sensory nerve has a frequency between about 1 Hz and about 200 Hz, an amplitude between about 1 mA and about 50 mA, and a pulse width between about 200 μS and about 1 mS with a current density less than 5 mA/cm².

16. The method of any one of the preceding Clauses, wherein stimulation energy delivered to the sensory nerve has a frequency between about 1 Hz and about 200 Hz, an amplitude between about 1 mA and about 50 mA, and a pulse width between about 200 uS and about 1 mS with a current density less than 2 mA/cm².

17. A device for delivering an electrical stimulus to a nerve proximate a pharynx of a human patient, the device comprising:
an elongated member having a proximal portion configured to be coupled to an energy source and a distal portion configured to be positioned at a treatment site within the pharynx; and
a conductive element carried by the distal portion of the elongated member and configured electrically couple to the energy source to deliver an electrical stimulus to a nerve at or proximate a wall of the pharynx at the treatment site, wherein the electrical stimulus is configured to improve a consciousness level of the patient.

18. The device of any one of the preceding Clauses, wherein the electrical stimulus is configured to improve at least one of an eye opening response, a verbal response, a motor response, or an autonomic response of the patient.

19. The device of any one of the preceding Clauses, wherein the electrical stimulus is configured to improve at least one of a cortical connectivity parameter, a cerebral metabolism parameter, a cortical excitability parameter, a clinical scoring system score, a brainstem reflex parameter, or a task-based fMRI parameter.

20. The device of any one of the preceding Clauses, wherein the nerve comprises an afferent nerve.

21. The device of any one of the preceding Clauses, wherein the nerve comprises a sensorimotor nerve

22. The device of any one of the preceding Clauses, wherein the nerve comprises the vagus nerve.

23. The device of any one of the preceding Clauses, wherein the nerve comprises one or more cranial nerves.

24. The device of any one of the preceding Clauses, further comprising an elongated shaft configured to be slidably positioned through a lumen of the elongated member.

25. The device of any one of the preceding Clauses, further comprising a retaining structure configured to releasably engage the elongated shaft to fix a position of the elongated shaft relative to the elongated member.

26. The device of any one of the preceding Clauses, wherein the elongated shaft has a proximal portion and a distal portion configured to be positioned within the patient's stomach, and wherein the elongated shaft is configured to deliver nutrients to the stomach.

27. A method comprising:
via a nasal or oral cavity, positioning an elongated member carrying a conductive element in a lumen of a pharynx of a human subject such that the conductive element is positioned at a first location;
applying electrical stimulation energy at the first location via the conductive element;
after applying the electrical stimulation energy at the first location, obtaining first data characterizing a consciousness parameter of the subject;
positioning the elongated member in the lumen such that the conductive element is positioned at a second location;
applying electrical stimulation energy at the second location via the conductive element;
after applying the electrical stimulation energy at the second location, obtaining second data characterizing the consciousness parameter of the subject; and
comparing the consciousness parameter of the first data to the consciousness parameter of the second data.

28. The method of any one of the preceding Clauses, further comprising, based on the comparison, determining whether the application of electrical stimulation energy at the first location or the second location results in a more favorable consciousness parameter.

29. The method of any one of the preceding Clauses, wherein obtaining the first data and/or obtaining the second data includes:
providing an afferent stimulus to the subject, and
in response to the afferent stimulus, detecting the consciousness parameter.

30. The method of any one of the preceding Clauses, wherein the consciousness parameter comprises at least one of a cortical connectivity parameter, a cerebral metabolism parameter, a cortical excitability parameter, a clinical scoring system score, a brainstem reflex parameter, or a task-based fMRI parameter.

31. The method of any one of the preceding Clauses, wherein the afferent stimulus comprises a sensory stimulus.

32. The method of any one of the preceding Clauses, wherein the afferent stimulus comprises transcranial magnetic stimulation.

33. The method of any one of the preceding Clauses, wherein obtaining the first data and/or obtaining the second data comprises obtaining electroencephalography (EEG) data from the subject.

34. The method of any one of the preceding Clauses, wherein obtaining the first data and/or obtaining the second data comprises obtaining imaging data from the subject, the imaging data comprising at least one of magnetic resonance imaging (MM) data, computed tomography (CT) data, or positron emission tomography (PET) data.

35. The method of any one of the preceding Clauses, wherein obtaining the first data and/or obtaining the second data comprises detecting a biomarker.

36. The method of any one of the preceding Clauses, further comprising:
obtaining first baseline data characterizing a consciousness parameter of the subject before applying the electrical stimulation energy at the first location;
obtaining second baseline data characterizing a consciousness parameter of the subject before applying the electrical stimulation energy at the second location;
comparing the consciousness parameter associated with the first data to the consciousness parameter associated with the first baseline data; and
comparing the consciousness parameter associated with the second data to the consciousness parameter of the second baseline data.

37. A method comprising:
obtaining baseline data characterizing a consciousness parameter of a subject;
delivering electrical stimulation energy at a first location within a pharynx of the subject;
during and/or after delivering electrical stimulation energy at the first location, obtaining first data characterizing the consciousness parameter;
delivering electrical stimulation energy at a second location within the pharynx;
during and/or after delivering electrical stimulation energy at the second location, obtaining second data characterizing the consciousness parameter; and
based on at least two of the baseline data, the first data, or the second data, identifying which of the first or second locations is a preferred treatment location for delivering electrical stimulation energy.

38. The method of any one of the preceding Clauses, wherein obtaining the baseline data comprises obtaining first baseline data, the method further comprising, after delivering electrical stimulation energy at the first location and before delivering electrical stimulation energy at the second location, obtaining second baseline data characterizing the consciousness parameter.

39. The method of any one of the preceding Clauses, wherein the consciousness parameter comprises a score on a clinical scoring system.

40. The method of any one of the preceding Clauses, wherein the score on the clinical scoring system comprises at least one of a Coma Recovery Scale—Revised score, a Glasgow Coma Scale score, a Full Outline of UnResponsiveness score, a Motor Behavior Tool—Revised score, or a World Federation of Neurological Societies score.

41. The method of any one of the preceding Clauses, wherein the consciousness parameter comprises a brainstem function.

42. The method of any one of the preceding Clauses, wherein the brainstem function comprises at least one of a pupillary reflex, a corneal reflex, an oculocephalic reflex, an oculovestibular reflex, a cough reflex, or a gag reflex.

43. The method of any one of the preceding Clauses, wherein the consciousness parameter comprises a feature of an EEG signal.

44. The method of any one of the preceding Clauses, wherein the feature of the EEG signal comprises at least one of a power, a frequency, a correlation between electrodes, or an EEG complexity.

45. The method of any one of the preceding Clauses, wherein the consciousness parameter comprises a cortical connectivity parameter.

46. The method of any one of the preceding Clauses, wherein the cortical connectivity parameter comprises at least one of a permutation entropy (PE), symbolic mutual information (SMI), weighted Symbolic Mutual Information (wSMI), weighted Phase Lag Index (wPLI), symmetric uncertainty, phase locking value (PLV), phase lag index (PLI), Granger Causality (GC), or a Perturbational Complexity Index (PCI).

47. The method of any one of the preceding Clauses, wherein the consciousness parameter comprises a feature of at least one of magnetic resonance imaging (MM) data, functional MM (fMRI) data, diffusion tensor imaging (DTI) data, computed tomography (CT) data, or positron emission tomography (PET) data.

48. The method of any one of the preceding Clauses, wherein the consciousness parameter comprises at least one of an ability to follow a command, an ability to speak or vocalize, a purposeful movement, or an ability to open the eyes.

49. The method of any one of the preceding Clauses, wherein the consciousness parameter comprises a cerebral metabolism parameter.

50. The method of any one of the preceding Clauses, wherein the consciousness parameter comprises a biomarker.

51. The method of any one of the preceding Clauses, wherein the biomarker comprises at least one of neuron-specific enolase, serum Tau, or neurofilament light chain.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 is a fragmentary sagittal view of a pharynx of a human patient.

FIG. 2 depicts an electrical stimulation system in accordance with several embodiments of the present technology.

FIGS. 3A and 3B depict an electrical stimulation device transnasally inserted into a pharynx of a patient in accordance with several embodiments of the present technology.

FIG. 4 is a flow diagram of an example process for identifying a treatment location in accordance with the present technology.

### DETAILED DESCRIPTION

The present technology relates to electrical stimulation devices and associated systems and methods. In particular embodiments, the present technology comprises electrical stimulation devices configured to electrically stimulate an upper gastrointestinal tract of a patient to treat one or more conditions. Some embodiments, for example, are directed to performing pharyngeal electrical stimulation (PES) to treat disorders of consciousness.

### I. Anatomy and Physiology

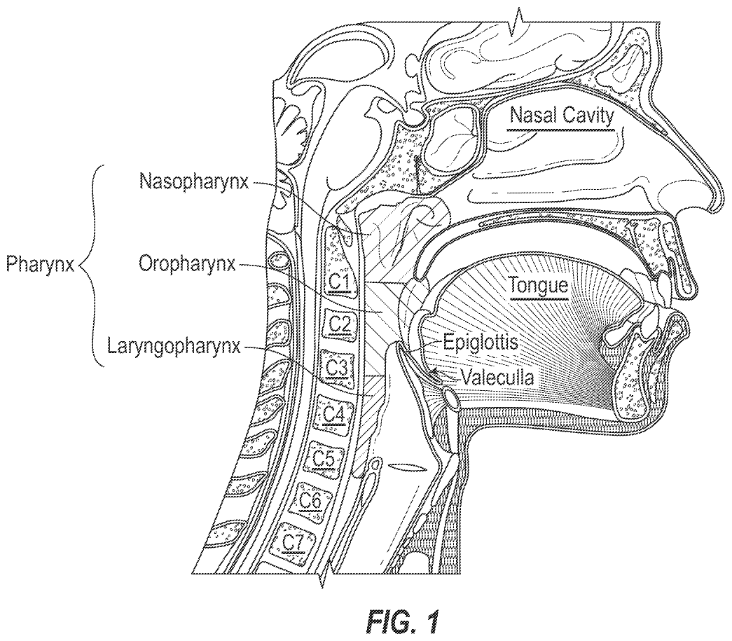

The pharynx is the part of the digestive system situated posterior to the nasal and oral cavities and posterior to the larynx. It is therefore divisible into nasal, oral, and laryngeal parts: the (1) nasopharynx, (2) oropharynx, and (3) laryngopharynx. With reference to FIG. 1, the pharynx extends from the base of the skull down to the inferior border of the cricoid cartilage (around the C6 vertebral level), where it becomes continuous with the esophagus. Its superior aspect is related to the sphenoid and occipital bones and the posterior aspect to the prevertebral fascia and muscles as well as the upper six cervical vertebrae. The pharynx is a fibromuscular tube lined by mucous membrane.

The pharynx is the common channel for deglutition (swallowing) and respiration, and the food and air pathways cross each other in the pharynx. In the anesthetized patient, the passage of air through the pharynx is facilitated by extension of the neck.

The nasopharynx, at least in its anterior part, may be regarded as the posterior portion of the nasal cavity, with which it has a common function as part of the respiratory system. The nasopharynx communicates with the oropharynx through the pharyngeal isthmus, which is bounded by the soft palate, the palatopharyngeal arches, and the posterior wall of the pharynx. The isthmus is closed by muscular action during swallowing. The choanae are the junction between nasopharynx and the nasal cavity proper.

A mass of lymphoid tissue, the nasopharyngeal tonsil is embedded in the mucous membrane of the posterior wall of the nasopharynx. Enlarged nasopharyngeal tonsils are termed “adenoids” and may cause respiratory obstruction. Higher up, a minute pharyngeal hypophysis (resembling the adenohypophysis) may be found.

The oropharynx extends inferiorward from the soft palate to the superior border of the epiglottis. It communicates anteriorly with the oral cavity by the faucial (oropharyngeal) isthmus, which is bounded superiorly by the soft palate, laterally by the palatoglossal arches, and inferiorly by the tongue. This area is characterized by a lymphatic ring composed of the nasopharyngeal, tubal, palatine, and lingual tonsils.

The mucous membrane of the epiglottis is reflected onto the base of the tongue and onto the lateral wall of the pharynx. The space on each side of the median glosso-epiglottic fold is termed the epiglottic vallecula.

The laryngopharynx extends from the superior border of the epiglottis to the inferior border of the cricoid cartilage, where it becomes continuous with the esophagus. Its anterior aspect has the inlet of the larynx and the posterior aspects of the arytenoid and cricoid cartilages. The pyriform sinus, in which foreign bodies may become lodged, is the part of the cavity of the laryngopharynx situated on each side of the inlet of the larynx.

A. Muscles

The pharynx consists of four coats of muscles, from within outward: (1) a mucous membrane continuous with that of the auditory tubes and the nasal, oral, and laryngeal cavities; (2) a fibrous coat, that is thickest in its superior extent (pharyngobasilar fascia) and that forms a median raphe posteriorly; (3) a muscular coat, described below; and (4) a fascial coat (buccopharyngeal fascia) covering the outer surface of the muscles.

The wall of the pharynx is composed mainly of two layers of skeletal muscles. The external, circular layer comprises three constrictors. The internal, chiefly longitudinal layer consists of two levators: the stylopharyngeus and the palatopharyngeus.

The chief action in which the muscles of the pharynx combine is deglutition (or swallowing), a complicated, neuromuscular act whereby food is transferred from (1) the mouth through (2) the pharynx and (3) the esophagus to the stomach. The pharyngeal stage is the most rapid and most complex phase of deglutition. During swallowing, the nasopharynx and vestibule of the larynx are sealed but the epiglottis adopts a variable position. Food is usually deviated laterally by the epiglottis and ary-epiglottic folds into the piriform recesses of the laryngopharynx, lateral to the larynx. The pharyngeal ridge is an elevation or bar on the posterior wall of the pharynx inferior to the level of the soft palate; it is produced during swallowing by transverse muscle fibers.

B. Innervation and Blood Supply

The motor and most of the sensory supply to the pharynx is by way of the pharyngeal plexus, which is formed by the pharyngeal branches of the vagus and glossopharyngeal nerves and also by sympathetic nerve fibers. The motor fibers in the plexus are carried by the vagus (although they likely represent cranial accessory nerve components) and supply all the muscles of the pharynx and soft palate except the stylopharyngeus (supplied by cranial nerve IX) and tensor veli palatini (supplied by cranial nerve V). The sensory fibers in the plexus are from the glossopharyngeal nerve, and they supply the greater portion of all three parts of the pharynx. The pharynx is supplied by branches of the external carotid (ascending pharyngeal) and subclavian (inferior thyroid) arteries.

The vagus nerve (cranial nerve X) is the longest cranial nerve in the body, containing both motor and sensory functions in both the afferent and efferent regards. The pharyngeal branch of the vagus nerve arises from the inferior ganglion of the vagus nerve and contain both sensory and motor fibers. A primary function of the vagus nerve is afferent and involves communicating sensory information to the brain. About 80% of the vagus nerve is composed of afferent fibers, projecting to the nucleus tractus solitarii (NTS) in the medulla, before being relayed further to other brainstem nuclei and higher-order structures, including the thalamus, hippocampus, amygdala, and insula. The vagus nerve also controls the superior, middle, and inferior pharyngeal constrictor muscles in conjunction with the glossopharyngeal nerve.

### II. Devices, Systems, and Methods for Treating Disorders of Consciousness

Injury and/or disease of the brain can result in withdrawal of excitatory synaptic activity across the cerebral cortex and resulting in disorders of consciousness. The downregulation of neuronal firing rates can be caused by direct structural loss of inputs or reduced input to neocortical and thalamic neurons caused by functional withdrawal of excitatory neurotransmission and/or disruption/disconnection of afferent neuronal inputs (e.g., deafferentation). Thus, without being bound by theory, it is believed that functional reemergence of the brainstem's ascending arousal network via increasing afferent feedback to higher brain centers improves wakefulness and awareness. Reafferentation, or the reestablishment of afferent neuronal inputs, can occur during the process of neuronal plasticity and contributes to the recovery of disorders of consciousness.

The present inventors have discovered that stimulation of the vagus nerve can induce neural plasticity and increase neural activity, which can improve consciousness. For example, stimulation of the vagus nerve is believed to induce and accelerate reorganization of the thalamo-cortical network, increase functional connectivity of the fronto-parietal network, enhance information sharing within the centro-posterior network, increase metabolism in the forebrain, thalamus, and reticular formation, and enhance and accelerate neuronal firing in the locus coeruleus. Additionally, vagus nerve stimulation is believed to increase the release of excitatory neurotransmitters such as acetyl choline, norepinephrine, orexins, glutamate, dopamine, serotonin, 5-hydroxytryptamine, and histamine.

The treatment devices of the present technology are configured to electrically stimulate the vagus nerve to produce afferent nerve signals that are transmitted to higher brain centers and induce functional cortical reorganization and increased cortical activity. The stimulation conditions and the afferent signals arising are designed to target, excite, and/or cause functional reorganization of specific areas in the brain associated with consciousness (e.g., the upper brainstem, the thalamus, the posterior cingulate cortex, etc.). As such, the stimulation energy delivered by the devices of the present technology can induce and accelerate neuroplasticity and increase neuronal activity to improve consciousness.

Some existing approaches for stimulating the vagus nerve rely on invasive or transcutaneous stimulation. Invasive vagus nerve stimulation has been used to treat several neurological disorders but requires a stimulator to be implanted in a patient's body and is associated with many perioperative risks. Transcutaneous vagus nerve stimulation involves the application of electrical currents through surface electrodes at the auricular branch of the vagus nerve located at the ear. However, transcutaneous vagus nerve stimulation is only able to stimulate small and peripheral branches of the vagus nerve, which may lead to limited efficacy. Moreover, neither invasive nor transcutaneous vagus nerve stimulation facilitate care of the patient secondary to treatment of the disorder of consciousness (e.g., feeding, hydration, medication, etc.). As previously noted, patients suffering from disorders of consciousness are often unable to safely feed themselves and require a feeding tube to provide nutrition. The devices of the present technology are configured to noninvasively stimulate the vagus nerve and, at least in some embodiments, also provide nutrition to a patient. Additionally, the devices of the present technology can be easier to implement, more convenient, more effective, and less invasive than existing vagus nerve stimulators.

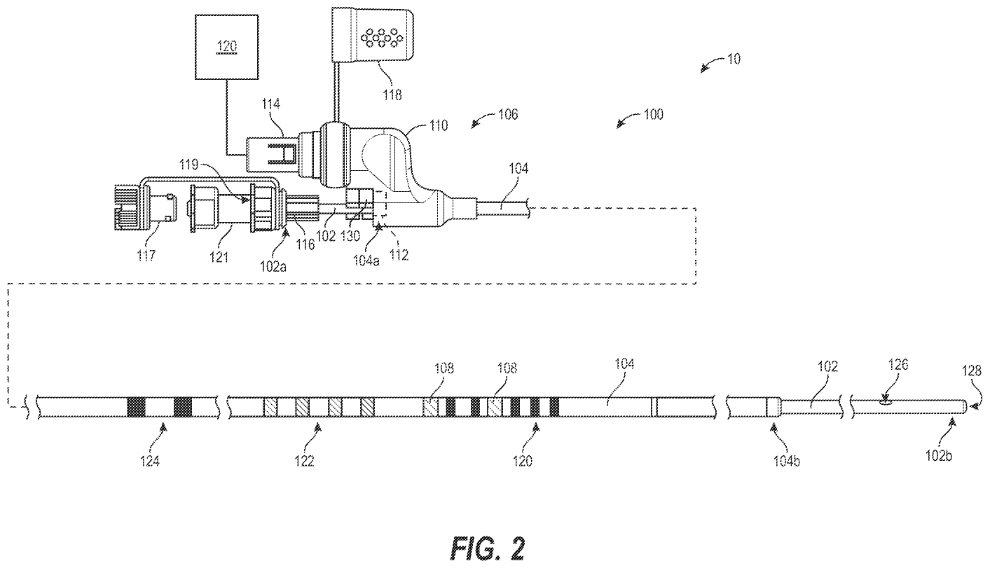

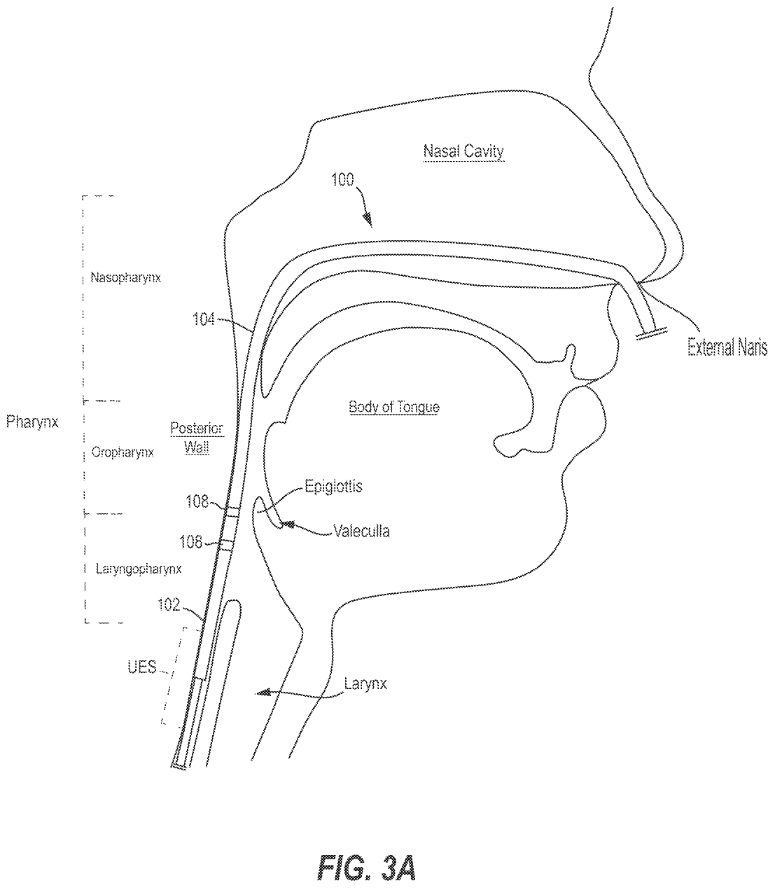

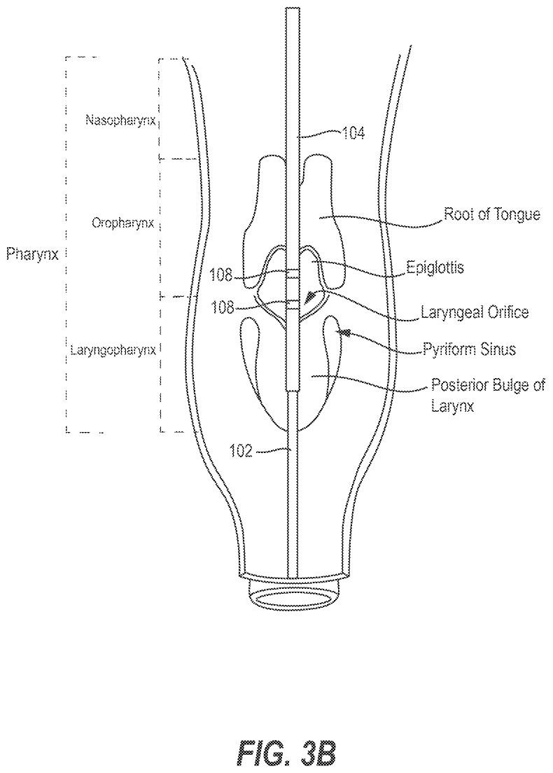

FIG. 2 depicts a treatment system 10 for improving a patient's level of consciousness in accordance with several embodiments of the present technology. The system 10 may comprise a device 100 configured to provide intraluminal electrical stimulation to a patient suffering from a medical condition, and a current generator 120 configured to be electrically coupled to the device 100. The device 100 can include a handle assembly 106, a first elongated shaft 102 (or “first shaft 102”), and a second elongated shaft 104 (or “second shaft 104”) configured to slidably receive the first shaft 102 therethrough. In some embodiments, the system 10 does not include the first shaft 102. The first shaft 102 can be a flexible tube configured to deliver nutrients to the patient. For example, in some embodiments the first shaft 102 comprises a nasogastric feeding tube. Each of the first and second shafts 102, 104 have a proximal portion 102a and 104a, respectively, coupled to the handle assembly 106, and a distal portion 102b and 104b, respectively, configured to be positioned within an upper gastrointestinal tract of the patient. As depicted in FIGS. 3A and 3B, when the device 100 is inserted into the patient, the distal portion 102b of the first shaft 102 can be configured to be positioned within the patient's stomach, and the distal portion 104b of the second shaft 104 is configured to be positioned at a treatment site within the patient's pharynx. In some embodiments, the distal portion 102b of the first shaft 102 can be configured to be positioned within the patient's intestines or pharynx. The device 100 further includes one or more conductive elements 108 carried by the distal portion 104b of the second shaft 104 and configured to deliver stimulation energy to a nerve proximate the treatment site. In some embodiments, the device 100 does not include the first shaft 102 and only includes the second shaft 104.

As shown in FIG. 2, the handle assembly 106 can include a hub 110 and one or more connectors and/or accessory components configured to be removably coupled to the hub 110. The hub 110 can include a housing having a first portion fixedly coupled to the proximal portion 104a of the second shaft 104, and a second portion configured to house one or more electrical components. In some embodiments, the second portion of the housing is positioned laterally of the first portion of the housing. The hub 110 may further include an electrical connector 114 at the second portion that provides an electrical interface between the second shaft 104 and the current generator 120.

In some embodiments, the device 100 includes a connector 116 coupled to the proximal portion 102a of the first shaft 102 and having one or more ports, such as port 119, configured to be coupled to one or more accessory devices or systems. For example, the port 119 may be configured to be releasably coupled to an enteral feeding set (not shown) for delivering nutrients through the first elongated shaft 102 into the patient's gastrointestinal tract. Additionally or alternatively, the port 119 can be configured to be releasably coupled to a guidewire assembly. For example, the port 119 can be configured to receive a guidewire (not visible) therethrough to assist with inserting the device 100 into the patient. The guidewire assembly can include a guidewire grip 121 coupled to the proximal end portion of the guidewire. In some embodiments, the connector 116 includes one or more additional ports, such as a port configured to be fluidly coupled to a syringe or other fluid source and/or pressure source. The connector 116 may further comprise a cap 117 tethered to the connector 116 and configured to be secured over the port 119 when not in use.

As previously mentioned, the first shaft 102 can be configured to be inserted through a lumen of the second shaft 104. In use, the distal portion 102b of the first shaft 102 can be inserted into an opening at the proximal portion 104a of the second shaft 104 that is fixed to the hub 110. In some embodiments, the device 100 includes a sealing member (not visible) at the hub 110 that engages the first and second shafts 102, 104 to prevent fluid from within a patient being drawn up within a space between the first and second shafts 102, 104 by way of capillary action when the second shaft 104 is removed from the patient. The sealing member may also be configured to clean any matter off of the first shaft 102 as it is withdrawn from the patient.

The first shaft 102 can have an atraumatic distal tip for patient comfort and ease of inserting the first shaft 102 into the patient. The first shaft 102 can have an opening 128 at its distal end and/or one or more apertures 126 extending through the sidewall along the distal portion 102b of the first shaft 102. Nutrients can be dispersed from the first shaft 102 into the patient's gastrointestinal tract via the one or more apertures 126 and/or the opening 128.

Each of the conductive elements 108 may comprise an electrode, an exposed portion of a conductive material, a printed conductive material, and other suitable forms. In some embodiments, for example as shown in FIG. 1, the conductive elements 108 comprise a pair of ring electrodes configured to deliver bipolar stimulation energy. The conductive elements 108 can be crimped, welded, or otherwise adhered to an outer surface of the second shaft 104. In some embodiments, the conductive elements 108 comprise a flexible conductive material disposed on the second shaft 104 via printing, thin film deposition, or other suitable techniques. While the device 100 shown in FIG. 1 includes two conductive elements 108, in some embodiments the device 100 may include more or fewer than two conductive elements 108. For example, the device 100 may comprise a single conductive element 108 configured to generate a monopolar electric field. Such embodiments include a neutral or dispersive electrode electrically connected to the current generator 120 and positioned on the patient's skin. As used herein, “conductive elements” can refer to a single conductive element or multiple conductive elements. In some embodiments, the device 100 may include three or more conductive elements 108 spaced apart along a longitudinal axis of the second shaft 104.

The device 100 may include one or more conductive leads (not visible) extending between a proximal portion of the device 100, such as the hub 110, and the conductive elements 108. In some embodiments, for example, the conductive leads comprise two wires, each extending distally from the hub 110 through a channel (the same channel or different channels) in the second shaft 104 to a corresponding one of the conductive elements 108. The channel(s), for example, can extend longitudinally within a sidewall of the shaft 104. The conductive leads can be insulated along all or a portion of their respective lengths.

) In some embodiments, the device 100 is configured such that a position of the second shaft 104 can be fixed relative to a position of the first shaft 102 (or vice versa). The position of the first shaft 102 relative to the second shaft 104 may be adjusted prior to insertion of the device 100 into the patient. Once adjustment is complete, the relative positions of the first shaft 102 and second shaft 104 may be substantially fixed. For example, as shown in FIG. 1, the proximal portion 100a of the device 100 can include a retaining structure 130 configured to be coupled to the hub 110 and/or the proximal portions 102a, 104a of one or both of the second shaft 104 and the first shaft 102. The retaining structure 130 can have a first portion on or through which the second shaft 104 and/or first shaft 102 may be movably or fixedly positioned, and a second portion moveable relative to the first portion. When the second portion is in an open position (as shown in FIG. 1), the first shaft 102 and the second shaft 104 can move longitudinally relative to one another. When the second portion is in a closed position (not shown), the longitudinal and/or radial positions of the first shaft 102 and the second shaft 104 are substantially fixed relative to one another.

The retaining structure 130 may be fixed to one of the first shaft 102 or the second shaft 104 and, at least in the open configuration, allow movement of the other of the first shaft 102 and the second shaft 104. In some embodiments, for example as shown in FIG. 1, the proximal portion of the second shaft 104 is fixed to the retaining structure 130 and the proximal portion of the first shaft 102 is slidably received by the first portion when the device 100 is assembled and the second portion is in an open position. When the second portion is in a closed position, the second portion engages the first shaft 102 and fixes the first shaft 102 relative to the second shaft 104, the hub 110, and/or the retaining structure 130.

In any case, the portion of the retaining structure 130 configured to engage the first and/or second shafts 102, 104 can comprise a high friction thermoplastic elastomer liner that engages the proximal portion of the first shaft 102 when the second portion is in the closed position. The liner can be configured to reduce the compressive force required to fix the first shaft 102 and thereby prevent pinching of the first shaft 102. Other suitable shapes, materials, positions, and configurations for the retaining structure 130 are possible. For example, the retaining structure 130 can comprise one or more magnets, a screw and threaded insert, a radial compression clip, and/or others to fix the proximal portion of the first shaft 102 to the retaining structure 130, the hub 110, and/or the second shaft 104.

As previously mentioned, the proximal portion of the device 100 and/or second shaft 104 is configured to be electrically coupled to a current generator 120 for delivering electric current to the conductive elements 108. The current generator 120, for example, can include a power source and a controller. The controller includes a processor coupled to a memory that stores instructions (e.g., in the form of software, code or program instructions executable by the processor or controller) for causing the power source to deliver electric current according to certain parameters provided by the software, code, etc. The power source of the current generator 120 may include a direct current power supply, an alternating current power supply, and/or a power supply switchable between a direct current and an alternating current. The current generator 120 can include a suitable controller that can be used to control various parameters of the energy output by the power source or generator, such as intensity, amplitude, duration, frequency, duty cycle, and polarity. Instead of or in addition to a controller, the current generator can include drive circuitry. In such embodiments, the current generator can include hardwired circuit elements to provide the desired waveform delivery rather than a software-based generator. The drive circuitry can include, for example, analog circuit elements (e.g., resistors, diodes, switches, etc.) that are configured to cause the power source to deliver electric current according to the desired parameters. For example, the drive circuitry can be configured to cause the power source to deliver periodic waveforms. In some embodiments, the drive circuitry can be configured to cause the power source to deliver a unipolar square wave.

The current generator 120 may be configured to provide a stimulation energy to the conductive elements 108 that has an intensity, amplitude, duration, frequency, duty cycle, and/or polarity such that the conductive elements 108 apply an electric field at the treatment site that promotes neuroplasticity in the areas of the brain associated with consciousness. Without being bound by theory, the application of stimulation energy to the vagal nerve is believed to induce and accelerate functional reorganization of higher brain centers and increase cortical activity, leading to improvements in wakefulness and awareness.

In some embodiments, the stimulation intensity is based, at least in part, on one or more patient-specific thresholds measured prior to treatment. For example, the stimulation intensity can be based, at least in part, on a patient's sensory threshold, which is the lowest (or near lowest) level of stimulation that is detectable by the patient. For patients retaining little or no ability to communicate with the person or machine administering the test (such as a patient in a coma), the sensory threshold can be determined based on an autonomic response of the patient, such as heart rate, respiratory rate, pupillary response, etc. For less impaired patients that are wakeful but have impairments to their awareness (e.g., confusion, disorientation, trouble with language but still able to make noise, etc.), the sensory threshold may be determined based on a verbal response (e.g., words, grunting, etc.), body movements (e.g., hand movements, foot movements, etc.). Additionally or alternatively, the stimulation intensity can be based, at least in part, on an upper threshold, which is the highest (or near highest) level of stimulation that can be tolerated by the patient. In some embodiments, the controller automatically calculates the correct stimulation level from the sensory threshold and the upper threshold. For some patients it may not be possible to distinguish between the sensory threshold and the upper threshold, or even measure one or both of the sensory threshold and the upper threshold. In such cases, the stimulation intensity can be determined by other methods, such as by selecting a stimulation program used for patients with similar characteristics to the non-responsive patient, such as age, sex, weight, clinical presentation, etc.

The current generator 120 can provide, for example, a current of about 1 mA to about 50 mA, about 1 mA to about 40 mA, about 1 mA to about 30 mA, about 1 mA to about 20 mA, or about 1 mA to about 10 mA, at a frequency of about 1 Hz to about 50 Hz, about 1 Hz to about 40 Hz, about 1 Hz to about 30 Hz, about 1 Hz to about 20 Hz, about 1 Hz to about 10 Hz, about 2 Hz to about 8 Hz, about 1 Hz, about 2 Hz, about 3 Hz, about 4 Hz, about 5 Hz, about 6 Hz, about 7 Hz, about 8 Hz, about 9 Hz, or about 10 Hz, and having a pulse width of about 100 μS to about 1 mS, about 150 μS to about 950 about 200 μS to about 900 about 250 μS to about 850 about 300 μS to about 800 about 350 μS to about 750 about 400 μS to about 700 about 450 μS to about 650 about 500 μS to about 600 or about 200 μS to about 1 mS. An energy of a pulse delivered can be no greater than 3 J, no greater than 2 J, or no greater than 1 J. A current density delivered can be no greater than 5 mA/cm2, no greater than 4 mA/cm2, no greater than 3 mA/cm2, no greater than 2 mA/cm2, or no greater than 1 mA/cm2. A total energy delivered during a treatment session can be no greater than 8 J, no greater than 7 J, no greater than 6 J, no greater than 5 J, no greater than 4 J, no greater than 3 J, no greater than 2 J, or no greater than 1 J.

The current generator 120 may also be configured to monitor contact quality between the conductive elements 108 and patient tissue during treatment set up/optimization and throughout the treatment process. In some embodiments, the current generator 120 records and stores patient information and includes a USB port to enable downloading of patient data. The current generator may include a touch screen user interface and software to guide a user through the treatment process.

FIGS. 3A and 3B are a fragmentary sagittal view and an open posterior view, respectively, of a patient's throat with a device 100 of the present technology inserted through the patient's nasal cavity into the pharynx. As shown, during a given treatment, the distal portion of the second shaft 104 is positioned at a treatment site in the pharynx. The conductive elements 108 can be located apposition with the posterior pharyngeal wall, the anterior pharyngeal wall, the medial pharyngeal wall, and/or the lateral pharyngeal wall. In some embodiments, the conductive elements 108 are positioned within a treatment window along the oropharynx and/or the laryngopharynx. In some embodiments, for example, the conductive elements 108 are positioned at a location that is approximately equivalent to the junction between the C3 and C4 cervical vertebrae. In any case, when energy is delivered to the conductive elements 108 by the current generator 120, the conductive elements 108 emit an electric field that flows through the wall of the pharynx and reaches the portion of the vagus nerve proximate the wall of the pharynx.

In some embodiments, the first and/or second shafts 102, 104 can comprise one or more indicators (such as indicators 120, 122, and 124 in FIG. 1) configured to facilitate insertion and positioning of the device 100 within the patient. For example, the indicator can comprise one or more visual markings that, when viewed through the patient's oral cavity, indicate the conductive elements 108 are properly positioned or that the second shaft 104 (and/or conductive elements 108) should be inserted further or withdrawn. In some embodiments, the indicator comprises one or more circumferential markings (such as one or more colored bands) printed on the second shaft 104.

When the conductive elements 108 are in a desired position, stimulation energy is delivered to the treatment site. In some embodiments, the delivered current is a unipolar square wave having a total energy per pulse of less than 3 J, less than 2 J, less than 1 J, about 3 J, about 2 J, or about 1 J. Each treatment session can have a duration between 5 minutes and 20 minutes. For example, the treatment session can have a duration of 10 minutes. In any case, a total energy delivered during the treatment session can be less than 8 J, less than 7 J, less than 6 J, less than 5 J, less than 4 J, less than 3 J, less than 2 J, less than 1 J, about 8 J, about 7 J, about 6 J, about 5 J, about 4 J, about 3 J, about 2 J, or about 1 J. In some embodiments, a patient can undergo a single treatment per day over the course of multiple days of treatment. For example, a patient can undergo one treatment session per day for three to six consecutive days. In some embodiments, the patient may undergo multiple treatment sessions per day and/or per week. Still, other stimulation energy parameters treatment session frequencies are possible.

The electrical stimulation of the present technology is configured to excite central nervous system regions associated with consciousness. The electrical stimulus is configured to treat a disorder of consciousness within a single treatment session and/or over multiple treatment sessions. As used herein, “treat” can mean complete or partial treatment, and can include improvements in the wakefulness and/or awareness. The position of the conductive elements within the pharynx when delivering the electrical stimulation energy influences the efficacy and safety of PES to treat disorders of consciousness. For example, if the conductive elements are positioned too far superiorly (e.g., proximally) within the pharynx, the trigeminal nerve or the facial nerves may be stimulated, which may in turn cause undesirable off-target effects such as facial pain or jaw chattering. If the conductive elements are positioned too far inferiorly (e.g., distally), delivery of the electrical stimulation energy may unintentionally modify a patient's cardiac activity and/or motor activity of the patient's upper esophageal sphincter. Moreover, such positioning may make it challenging to obtain sufficient contact between the conductive elements and the pharyngeal mucosa, which may limit efficacy of the treatment. Because the density, depth, and/or types of nerves vary at different locations along the upper respiratory and gastrointestinal tracts, delivering energy at one location can be highly efficacious while delivering energy at another location as close as a centimeter away from the first location can provide only marginal therapeutic benefits.

To address the foregoing challenges, the present technology includes methods for identifying a treatment location for delivering stimulation energy to improve a level of consciousness. The method can comprise applying electrical stimulation energy at one or more locations in an upper gastrointestinal tract of a subject and, during and/or after applying the energy at one or more of the locations, evaluating one or more parameters associated with the subject's consciousness. In some embodiments, the preferred treatment location is the location which, when stimulated, produces the greatest change in a consciousness parameter(s) and/or the most favorable consciousness parameter(s).

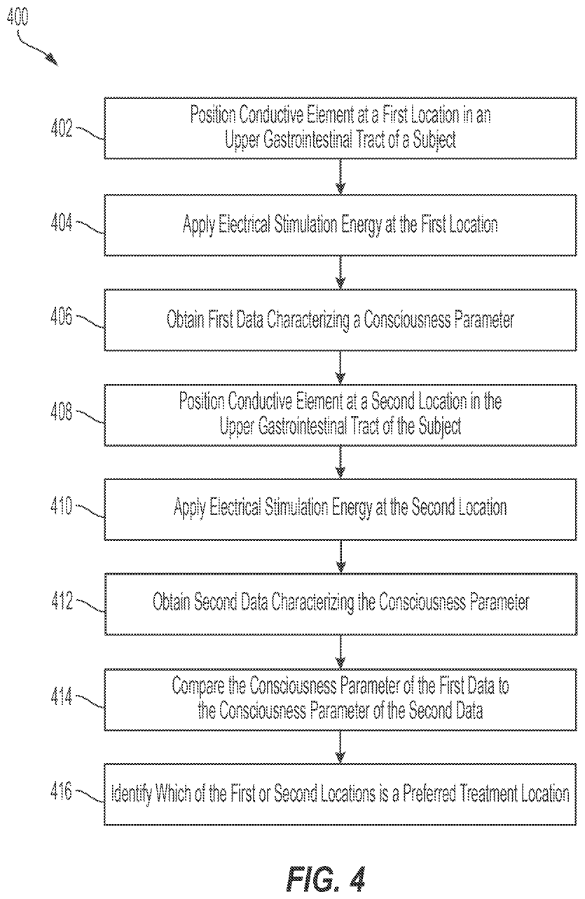

FIG. 4 is a flow diagram of an example process 400 for identifying a treatment location for PES utilizing the device 100 of the present technology. As described in greater detail herein, the process 400 can include positioning the conductive elements 108 at one or more potential treatment locations in an upper gastrointestinal tract (e.g., a pharynx, etc.) of a subject, applying electrical stimulation energy at each of the potential treatment locations, and obtaining data characterizing a consciousness parameter of the subject associated with stimulation of each potential treatment location. The data characterizing the consciousness parameter can be obtained prior to, during, and/or after applying the electrical stimulation energy at a potential treatment location. Obtaining the data characterizing the consciousness parameter can include, for example, conducting a clinical scoring examination, conducting a clinical examination of the subject, conducting imaging of the subject's central nervous system, conducting electrophysiology examinations of the subject, and/or obtaining chemical biomarkers from the subject. In some embodiments, the process 400 includes comparing the consciousness parameters associated with each potential treatment location and, based on the comparison, identifying which of the potential treatment locations is a preferred treatment location for delivering electrical stimulation energy to treat disorders of consciousness.

In some embodiments, the process 400 can identify a preferred treatment location that is associated with the most favorable consciousness parameter and/or change in consciousness parameter amongst a group of subjects. Additionally or alternatively, any of the processes disclosed herein can be performed with a single subject such that a subject specific preferred treatment location is identified. The human subject(s) may or may not be currently or previously suffering from disorders of consciousness.

As shown in FIG. 4, the process 400 can begin at block 402 with positioning the conductive elements 108 in the upper gastrointestinal tract of a human patient at a first location. The conductive elements 108 can be positioned within a pharynx of the patient. The first location can be along the nasopharynx, the oropharynx, or the laryngopharynx. In some embodiments, the conductive elements 108 are carried by the second shaft 104, as detailed herein. In other embodiments, the conductive elements 108 are carried by an elongated member that does not include a lumen extending therethrough. For ease of explanation, the process 400 will be described with reference to the second shaft 104 but equally applies to conductive elements 108 carried by the aforementioned elongated member. The second shaft 104 can be inserted into the lumen of the upper gastrointestinal tract via a nasal cavity of the subject (see FIGS. 3A and 3B, for example) or an oral cavity of the subject. Additionally or alternatively, the second shaft 104 can be inserted into the lumen the upper gastrointestinal tract of the subject via transcutaneous, transmuscular, and/or percutaneous insertion.

In some embodiments, positioning the conductive elements 108 at the first location can comprise evaluating a position of the conductive elements 108 once the second shaft 104 has been inserted and, optionally, repositioning the conductive elements 108 until the conductive elements 108 are positioned at the first location. In some embodiments, the second shaft 104 comprises one or more indicators configured to facilitate positioning of the conductive elements 108 at an intended location (e.g., the first location, etc.). For example, the indicator can comprise one or more visual markings that, when viewed through the patient's oral cavity, indicate if the conductive elements 108 are properly positioned or if the second shaft 104 and/or conductive elements 108 should be inserted further or withdrawn. In some embodiments, the indicator comprises one or more circumferential markings (such as one or more colored bands) on the second shaft 104.

The process 400 can proceed at block 404 with applying electrical stimulation energy at the first location via the conductive elements 108. As shown at block 406 in FIG. 4, the process 400 can include obtaining first data characterizing a consciousness parameter of the subject. The process 400 can include obtaining the first data during and/or after applying the electrical stimulation energy at the first location. Although not depicted in FIG. 4, the process 400 can include obtaining first baseline data characterizing the consciousness parameter prior to applying the electrical stimulation energy at the first location. According to various embodiments, the processes for obtaining the first data and the first baseline data can be the same. In some embodiments, the processes for obtaining the first data and the first baseline data can be different.

The consciousness parameter can comprise a direct or an indirect parameter of a subject's wakefulness and/or awareness. In some embodiments, the consciousness parameter comprises a score from a clinical scoring system (e.g., the Glasgow Coma Scale, the Full Outline of UnResponsiveness, the Coma Recovery Scale—Revised, etc.), a result of a brainstem function test (e.g., pupillary light reflex, corneal reflex, oculocephalic reflex, oculovestibular reflex, cough reflex, gag reflex, etc.), a result of brain imaging (e.g., computed tomography (CT), magnetic resonance imaging (MRI), diffusion tensor imaging (DTI), etc.), a result of an electrophysiological technique (e.g., electroencephalogram (EEG), electromyography (EMG), electrocardiography (ECG), functional magnetic resonance imaging (fMRI), positron emission tomography (PET), etc.), and/or a chemical biomarker (e.g., phosphatidylcholine, arachidonic acid, etc.). A consciousness parameter derived from EEG techniques can include, but is not limited to, permutation entropy (PE), symbolic mutual information (SMI), weighted Symbolic Mutual Information (wSMI), weighted Phase Lag Index (wPLI), symmetric uncertainty, phase locking value (PLV), phase lag index (PLI), Granger Causality (GC), Perturbational Complexity Index (PCI), and/or others. In some embodiments, a consciousness parameter can be obtained in response to an afferent stimulus. For example, transcranial magnetic stimulation (TMS) can be applied to a patient's cortex (e.g., at locations associated with consciousness) and EEG can be used to measure electrical responses of the cortex locally and at distant sites, enabling the study of cortical excitability under the site of stimulation and long-range cortical connectivity. Additionally or alternatively, motor evoked potentials (MEPs) can be evaluated in response to TMS to determine cortical excitability of motor cortices. In some embodiments, an afferent stimulus for obtaining the data can comprise a sensory (e.g., auditory, visual, tactile, etc.) stimulus. Obtaining data (e.g., first baseline data, first data, etc.) characterizing the consciousness parameter can include performing a clinical examination using a clinical scoring system and/or a brainstem function test, obtaining imaging of the subject's central nervous system, performing an electrophysiological technique on the subject, obtaining a subject's bodily fluid and performing analysis of the bodily fluid, and/or performing other diagnostic evaluations.

The process 400 can include applying electrical stimulation energy at multiple locations in the subject's upper gastrointestinal tract and evaluating a consciousness parameter associated with application of the electrical stimulation energy at each of the locations. For example, as shown in FIG. 4, the process 400 can include positioning the conductive elements 108 at a second location in the upper gastrointestinal tract of the subject (block 408), applying electrical stimulation energy at the second location (block 410), and obtaining second data characterizing the consciousness parameter of the subject (block 412). The second location can be spaced apart from the first location. Any of the blocks 408, 410, 412 can be similar to a corresponding block 402, 404, 406 as described with reference to the first location. The electrical stimulation energy applied at the second location at block 410 can have the same parameters as the electrical stimulation energy applied at the first location at block 404. Obtaining the second data can be similar to obtaining the first data. In some embodiments, the process 400 includes obtaining second baseline data prior to applying the electrical stimulation energy at the second location.

Obtaining the first data and/or obtaining the second data can occur immediately after applying the stimulation energy, about 5 minutes or more after applying the stimulation energy, about 15 minutes or more after applying the stimulation energy, about 30 minutes or more after applying the stimulation energy, about 60 minutes or more after applying the stimulation energy, about 90 minutes or more after applying the stimulation energy, or about 120 minutes or more after applying the stimulation energy.

The process 400 can include comparing the consciousness parameters of at least two of the first baseline data, the first data, the second baseline data, or the second data. For example, as shown at block 414, the process 400 can include comparing the consciousness parameter of the first data to the consciousness parameter of the second data. Additionally or alternatively, the process 400 can include comparing the consciousness parameter of the baseline first data to the consciousness parameter of the first data and/or comparing the consciousness parameter of the baseline second data to the consciousness parameter of the second data. Comparing the consciousness parameters can comprise determining a difference between the consciousness parameters. For example, comparing the consciousness parameters can include determining a first difference in EEG spectral measures associated with the baseline first data and the first data, determining a second difference in EEG spectral measures associated with the sec baseline second data and the second data, and identifying which of the first difference or the second difference is greater. Comparing the consciousness parameters can comprise comparing topographical map, datasets, point clouds, individual data points, etc. In any of the embodiments disclosed herein, comparing the consciousness parameters can comprise performing a statistical analysis.

As shown in FIG. 4, the process 400 can include identifying which of the first or second locations is a preferred treatment location for delivering electrical stimulation energy (block 416). In some embodiments, the preferred treatment location is the location associated with a consciousness parameter measurements, or combination of consciousness parameter measurements, that are associated with a greater improvement in level of consciousness. In some embodiments, identifying the preferred treatment location can comprise performing a weighted analysis of multiple consciousness parameters. In these and other embodiments, consciousness parameters with the greatest clinical impact can influence identification of the preferred treatment location to a greater degree than consciousness parameters with less clinical impact. For example, if a change in wSMI is weighted higher than a change in a cortical glucose uptake identified by PET imaging, the location associated with the greatest change in wSMI may be identified as the preferred treatment location, even if such location is not associated with the greatest change in cortical glucose uptake. In some embodiments, determining the weighting of the consciousness parameters may involve correlating neurodiagnostic parameters to functional outcome measures and applying greater weighting to neurodiagnostic parameters that are most strongly correlated to functional outcome measures.

Identifying the preferred treatment location can comprise evaluating additional considerations such as off-target effects, practical limitations, and/or other considerations. For example, identifying the preferred treatment location can comprise determining whether application of electrical stimulation energy at the first and/or second locations results in off-target effects such as unintentional motor activity (e.g., jaw chattering, hand twitching, etc.), discomfort, and/or pain. If application of electrical stimulation energy at one of the first and/or second locations results in off-target effects, the location may not be identified as the preferred treatment location. To determine whether application of electrical stimulation energy at the first and/or second locations results in off-target effects, a biological signal (e.g., EMG, ECG, EEG, etc.) can be measured from the subject's face, jaw, hand, heart, and/or other suitable location.

Additionally or alternatively, feedback from the subject and/or an operator performing one or more portions of the process can be obtained to evaluate additional considerations (e.g., off-target effects, practical limitations, etc.) associated with applying electrical stimulation energy at the first and second locations. For example, if an operator struggles to insert the second shaft 104 such that the conductive elements 108 are positioned at the first location with sufficient contact between the conductive elements 108 and the wall of the upper gastrointestinal tract, the process may identify that the first location is not the preferred treatment location. Moreover, if a subject experiences pain or discomfort when the conductive elements 108 are positioned at one of the first or second locations, the process can identify that the location is not the preferred treatment location.

Although FIG. 4 depicts a process evaluating two potential treatment locations (e.g., the first and second locations), any number of potential treatment locations (e.g., one, two, three, four, five, six, seven, eight, nine, ten, more than ten, etc.) can be evaluated. For example, a predetermined number of potential treatment locations can be evaluated. In some embodiments, the predetermined potential treatment locations can span a certain portion of the upper gastrointestinal tract and/or be positioned according to predetermined spacing. In various embodiments, the process 400 can repeat positioning the conductive elements 108 at the potential treatment location, applying electrical stimulation energy at the potential treatment location, and obtaining data characterizing a consciousness parameter of the subject (e.g., blocks 402-406, blocks 408-412) for each of the potential treatment locations. The process 400 can then proceed with comparing the consciousness parameters associated with each potential treatment location. Additionally or alternatively, the process 400 can comprise comparing the consciousness parameters associated with two or more potential treatment locations and based on the comparison, identify a preferred treatment location of the two or more potential treatment locations. The process 400 may continue iterating and comparing the consciousness parameters associated with additional potential treatment locations or may terminate. In some embodiments, the process 400 can terminate once a desired consciousness parameter has been obtained. For example, the process 400 can terminate once a location associated with a change in a consciousness parameter greater than a predetermined threshold has been identified.

The particular processes described herein are exemplary only and may be modified as appropriate to achieve the desired outcome. In various embodiments, other suitable methods or techniques can be used to identify a treatment location. Moreover, although various aspects of the methods disclosed herein refer to sequences of steps, in various embodiments the steps can be performed in different orders, two or more steps can be combined together, certain steps may be omitted, and additional steps not expressly discussed can be included in the process as desired.

At least some of the processes described herein can be performed on one computing device or a cluster of computing devices working in concert, or various processes can be performed by remote or distributed computing devices, with different steps being performed by different entities and/or different computing devices. For example, some or all of the processes described herein can be performed in a distributed computing environment in which tasks or modules are performed by remote processing devices, which are linked through a communication network (e.g., a wireless communication network, a wired communication network, a cellular communication network, the Internet, a short-range radio network (e.g., via Bluetooth)). In various embodiments, some or all of the processes described herein can be performed automatically. According to some embodiments, some of the processes described herein may rely at least in part on one or more inputs from a human operator such as a clinician or technician.

### CONCLUSION

Although many of the embodiments are described above with respect to systems, devices, and methods for electrically stimulating an upper gastrointestinal tract of a patient to treat disorders of consciousness, the technology is applicable to other applications and/or other approaches. For example, the device may be used to treat other conditions, or used to apply a different form of energy (such as ablation energy). Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

As used herein, the terms “generally,” “substantially,” “about,” and similar terms are used as terms of approximation and not as terms of degree, and are intended to account for the inherent variations in measured or calculated values that would be recognized by those of ordinary skill in the art. cm I/We claim:

1. A method substantially as disclosed herein.

2. A system substantially as disclosed herein.

3. A device substantially as disclosed herein.
